(19) United States
(12) Patent Application Publication
(10) Pub. No.: US 2025/0222219 A1
(43) Pub. Date: Jul. 10, 2025

(54) RESPIRATORY DEVICE AND METHOD WITH SPONTANEOUS RESPIRATORY PHASES

(71) Applicant: Hamilton Medical AG, Bonaduz (CH)

(72) Inventors: Federico Alexander Wadehn, Bonaduz (CH); Johannes Meyer, Chur (CH); Alessandro Beda, Rhäzüns (CH); Thomas Laubscher, Rhäzüns (CH); Dominik Novotni, Chur (CH)

(21) Appl. No.: 18/853,151

(22) PCT Filed: Apr. 3, 2023
(86) PCT No.: PCT/EP2023/058694

§ 371 Date: Oct. 1, 2024

(30) Foreign Application Priority Data
Apr. 5, 2022 (DE) 10 2022 108 124.9

(51) Int. Cl. A61M 16/00

(52) U.S. Cl. CPC A61M 16/024; A61M 2016/0021; A61M 2016/0039; A61M 2230/46

(57) ABSTRACT

A respiratory device including a breathing gas source assembly, a flow change device, a breathing gas line assembly, a sensor assembly, and a control device with a data memory, the control device being connected for signal transmission to the data memory and to the sensor assembly, the control device being designed, in a normal respiratory operation, not to bring about a machine-triggered breathing stroke starting from a previous breathing stroke before a predetermined normal time interval has elapsed, the control device being further designed to identify, on the basis of data from the sensor assembly, a spontaneous respiratory effort of the patient, the control device being further designed, if it has identified at least a predetermined plurality of spontaneous respiratory efforts of the patient within a predetermined monitoring period, to switch to a challenge respiratory operation, in which, instead of the normal interval, a challenge interval lasting for a longer time is used.

This application claims priority in PCT application PCT/EP2023/058694 filed Apr. 3, 2023, which claims priority in German Patent Application DE 10 2022 108 124.9 filed on Apr. 5, 2022, which are incorporated by reference herein.

The present invention concerns a ventilation device for a ventilation operation for mechanical administration of respiratory gas to a patient, where the ventilation device comprises:
A respiratory gas source arrangement which provides an inhalatory respiratory gas for administration to the patient,
A flow-modification device which is configured to generate and/or quantitively modify an inhalatory respiratory gas flow,
A respiratory gas line arrangement in order to convey the inhalatory respiratory gas flow from the respiratory gas source arrangement towards the patient,
A sensor arrangement which is configured to capture at least one state variable of the inhalatory respiratory gas flow and/or a mass flow of the inhalatory respiratory gas flow and/or a respiratory musculature activity of the patient,
A control device with a data store, where the control device is connected with the data store and with the sensor arrangement for signal transmission, and where the control device is configured for controlling the flow-modification device,

Where the control device is configured to actuate, in a normal ventilation operation, the flow-modification device starting from a preceding breath at or after expiration of a predetermined temporal normal gap interval for generating an inhalatory respiratory gas flow, in order to effect a machine-triggered following breath which immediately follows the preceding breath,

Where the control device is further configured to recognize, on the basis of data ascertained by the sensor arrangement, a spontaneous respiratory effort of the patient during the ventilation operation of the ventilation device.

The present invention furthermore concerns a ventilation method for mechanical administration of respiratory gas to a patient, where the ventilation method comprises the following steps:
Effecting a machine-triggered following breath which immediately follows a preceding breath by generating an inhalatory respiratory gas flow by means of a flow-modification device starting from the preceding breath after expiration of a predetermined temporal normal gap interval,
Capturing at least one state variable of the inhalatory respiratory gas flow and/or of a mass flow of the inhalatory respiratory gas flow and/or of a respiratory musculature activity of the patient through a sensor arrangement,
Recognizing a spontaneous respiratory effort of the patient during the ventilation operation of the ventilation device on the basis of data ascertained through the sensor arrangement.

### BACKGROUND OF THE INVENTION

Spontaneous respiration activity of an artificially ventilated patient, in particular if it is not recognized or ignored by the ventilation device, constitutes a fundamental problem of artificial ventilation since the spontaneous respiration activity and/or which is tantamount to it the spontaneous respiratory efforts of the patient disturbs the rhythmical course of the artificial ventilation through the ventilation device. Spontaneous respiration activity normally has as a consequence that more or less respiratory gas is administered than is set at the ventilation device or that the respiratory musculature is excessively stressed or even damaged. Spontaneous respiratory efforts of the patient are therefore also often referred to as ‘asynchronies’.

From US 2011/0213215 A1 there is known as a component of a weaning strategy for weaning an artificially ventilated patient from the mechanical ventilation, interruption of the conventional artificial ventilation through so-called spontaneous breathing trials (SBT). The known spontaneous breathing trials are started on the basis of pre-set configurations, input control commands, or through appropriate selection of an operating mode. During these spontaneous breathing trials the control device monitors the ventilation device key parameters of the ventilation, such as for example a ratio of the breathing frequency to the tidal volume, which is also referred to as ‘frequency-volume respiratory index’ or as ‘rapid shallow breathing index’.

Further key parameters can be at least one parameter out of: The spontaneous tidal volume, the spontaneous exhalation volume, CO₂ elimination levels, blood oxygen saturation levels, heart rate, and an estimated respiratory work of the patient.

From the capture of volume values of the spontaneous respiration, such as the spontaneous tidal volume and the spontaneous exhalation volume, it follows that during the known spontaneous breathing trial no supporting ventilation of the patient takes place, but rather it is simply observed to what extent the patient is able independently to perform spontaneous breathing.

The control device of the ventilation device known from US 2011/0213215 A1 ends a spontaneous breathing trial when a predetermined time has expired or when one of the key parameters is captured outside or at least for a predetermined duration outside a predetermined range of values.

From US 2013/0053717 A1 there is known an operating mode of a ventilation device which is meant to trigger spontaneous respiration of a patient not breathing spontaneously. To this end the mechanical ventilation is reduced in order to raise the CO₂ partial pressure in the arteries of the patient above a particular threshold value. US 2013/0053717 A1 assumes the principle that when the arterial CO₂ partial pressure exceeds an individual threshold, it triggers spontaneous respiration in the patient.

From U.S. Pat. No. 9,078,984 B2 there are known a ventilation device and a ventilation method in which the ventilation frequency of a mechanical ventilation is brought up to the natural breathing frequency of a patient within a predetermined tolerance range in order to prevent the patient, through excessively large differences between the ventilation frequency of the mechanical ventilation and his natural, i.e. neural breathing frequency, beginning to fight against the mechanical ventilation.

From the SERVO-U ventilation system offered by the firm of Maquet GmbH (Rastatt, Germany) there is known a ventilation mode referred to as ‘automode’ in which the ventilation device changes over automatically between controlled and supported ventilation in order to improve the interaction between patient and ventilation device. When the patient in this ventilation mode exhibits spontaneous respiration activity, the ventilation device switches over immediately to a supporting mode in which the ventilation device supports the breath triggered by the spontaneous respiration activity through mechanical supply of inhalatory respiratory gas. If the patient no longer exhibits spontaneous respiration activity, the ventilation device changes back into the controlled mode and until further notice triggers breaths mechanically. Therefore the known ‘automode’ allows the patient to trigger breaths and supports the patient during the inhalation. Breaths triggered by the patient through spontaneous respiration activity have precedence over mechanically triggered breaths. If spontaneous respiratory efforts of the patient are absent, triggering takes place mechanically.

Unlike the state of the art described above, the following technical problem underlies the present application: Even in a ventilation mode which permits triggering of breaths through spontaneous respiratory efforts of the patient and supports such patient-triggered breaths through mechanical supply of inhalatory respiratory gas, such as for instance the ASV (adaptive support ventilation) mode in ventilation devices of the applicant, practically any spontaneous respiration of the patient is suppressed if his natural (neural) breathing frequency is slightly lower than the mechanical ventilation frequency. The normal gap interval of the normal ventilation mode as an inverse value of the purely mechanical ventilation frequency is then slightly shorter than the gap interval of the natural respiratory behavior of the patient. Should the patient succeed, through spontaneous respiratory effort, in triggering a breath, the following breath following this spontaneously triggered breath will be mechanically triggered since the normal gap interval of the ventilation device in normal ventilation operation is shorter than the natural or to be precise neural gap interval of the patient, observed between two spontaneous breaths. This leads to the situation that the patient is predominantly ventilated in a mechanically triggered manner, although he could trigger spontaneously. Most spontaneous respiratory efforts of the patient after a successful patient-triggered breath consequently fall in the inhalation phase of mechanically triggered breaths and are simply overridden by the control device.

The patient is thus on the one hand sufficiently active, cannot however trigger breaths himself through his spontaneous respiratory efforts. This can lead to a few drawbacks, such as for example to large transpulmonary pressure fluctuations and to tiredness of the diaphragm due to eccentric contractions, since the muscle is stretched under tensile stress. Likewise, it can result in the administration of excessive tidal volumes, double triggering events, large fluctuations in the administered inhalatory respiratory gas volume and consequently shortened exhalation phases, and quite generally asynchronicities. Further consequent from this, sensorially captured parameters, such as for example the tidal volume or the pulmonary time constant as the product of the pulmonary resistance and compliance, can be unreliable in terms of the captured quantities and lead to unstable control of the ventilation which relies on the sensorially captured parameter values.

### SUMMARY OF THE INVENTION

It is therefore the task of the present invention to provide a technical approach which allows giving an artificially ventilated but sufficiently spontaneously active patient the possibility of taking on the triggering control of his ventilation and not having his spontaneous respiratory efforts ignored and overridden by the control device of the ventilation device.

This task is solved according to the present invention in a ventilation device described at the beginning by having the control device configured to change over, when in a predetermined monitoring time period it has recognized at least one predetermined plurality of spontaneous respiratory efforts of the patient, into a challenge ventilation operation in which instead of the normal gap interval a challenge gap interval is used which is temporally longer compared with the normal gap interval, where in the challenge ventilation operation the control device does not effect a machine-triggered following breath which immediately follows a preceding breath before expiration of the challenge gap interval.

According to the present invention, this task is moreover solved in a ventilation method described in the beginning by the ventilation method comprising the following further steps:
When in a predetermined monitoring time period at least one predetermined plurality of spontaneous respiratory efforts of the patient has been recognized, changing over into a challenge ventilation operation in which instead of the normal gap interval a challenge gap interval is used which is temporally longer compared with the normal gap interval,
Effecting a machine-triggered following breath which immediately follows a preceding breath after expiration of the challenge gap interval.

The ventilation device according to the invention is preferably configured for performing the ventilation method according to the invention, which is why the following description of the ventilation device, which contains detailed aspects also of the ventilation method, is also a description of the ventilation method and its advantageous developments.

The fundamental idea of the present invention lies in first establishing that the artificially ventilated patient is sufficiently active, i.e. exhibits sufficient spontaneous respiration activity, in order to make it possible for him to take on the triggering of breaths through his spontaneous respiratory efforts as a triggering cause. To this end there should not suffice a single spontaneous respiratory effort of the patient, but rather the patient should exhibit in a predetermined monitoring time period a predetermined plurality of spontaneous respiratory efforts, such that a sufficient spontaneous respiration activity of the patient can be assumed.

If the sufficient spontaneous respiration activity of the patient is recognized, the normal gap interval is lengthened temporally such that on the one hand the natural or to be precise neural gap interval of the patient can be shorter than the lengthened gap interval referred to as ‘challenge gap interval’ to distinguish it from the normal gap interval and such that on the other hand a gap interval continues to be used in order to continue ventilating the patient if against expectation he durably does not exhibit sufficient spontaneous respiration activity.

The challenge ventilation operation referred to above differs from the normal ventilation operation preferably only through the lengthened gap interval between a breath and a machine-triggered following breath. The rest of the settings of the ventilation, such as in particular minute volume, preferably remain intact in order to ensure sufficient supply of the patient with inhalatory respiratory gas even with absent spontaneous respiration activity.

With the term ‘gap interval’ there is designated in the present application that temporal gap between a preceding breath and a following machine-triggered breath. After elapsing of the gap interval without interim spontaneous triggering, the control device necessarily triggers a following breath. With a spontaneously- or to be precise patient-triggered breath there begins anew the use of the gap interval as a waiting duration until the next machine-triggered breath. Consequently the circumstance that the following breath immediately follows the preceding breath means that between the preceding breath and the following breath there lies no further breath.

Breaths triggered by the control device are referred to in the present application as ‘machine-triggered’. Breaths triggered by a spontaneous respiratory effort of the patient are referred to in the present application as ‘patient-triggered’.

‘Triggering’ designates in the relevant professional world the triggering of an inhalation process. The triggering of an exhalation process or the modification of inhalation after exhalation is referred to in the professional world as ‘cycling’.

By ‘spontaneous respiration activity’ or ‘spontaneous respiratory effort’ there is designated an activity of the patient according to which the patient attempts to initiate a breath, in particular an inhalation process. On the patient's side this requires a muscular effort, normally in order to increase the volume of the chest cavity accommodating the lung or also of the neck and throat region. Such activity concerns initially directly only the initiation of a breath, not its complete independent execution. Therefore, in order to ensure sufficient supply of the patient with inhalatory respiratory gas, the control device is preferably configured to supply to the patient in the challenge ventilation operation, upon a spontaneous trigger of the patient, under predetermined ventilation conditions, inhalatory respiratory gas through the ventilation device. The challenge ventilation operation is, therefore, a supporting ventilation operation which ensures that sufficient inhalatory respiratory gas is supplied to the patient upon his spontaneous trigger. The ability of the patient to breathe completely independently, therefore, is not essential. It should, however, also not be ruled out that the patient can breathe independently. This case, however, is the great exception in the phase of an artificial ventilation which is relevant for the present application.

Since, as already described above, the normal ventilation operation and the challenge ventilation operation preferably differ only through the longer challenge gap interval, compared with the shorter normal gap interval, preferably the normal ventilation operation is also a ventilation operation which both triggers breaths mechanically and allows the spontaneous triggering of breaths by the patient and also in the case of breaths triggered spontaneously by the patient supplies to the patient a quantity of inhalatory respiratory gas through appropriate actuation of the flow-modification device. It should, however, not be ruled out that the normal ventilation operation is an exclusively controlling ventilation operation in which breaths are triggered exclusively mechanically.

The flow-modification device can comprise a fan or a pump whose rotational speed or whose delivery rate respectively is modifiable via the control device. Additionally or alternatively, the flow-modification device can comprise a valve whose flow-through cross-section is modifiable via the control device.

The respiratory gas source arrangement can be or comprise an intake port for ambient air as respiratory gas. Additionally or alternatively, the respiratory gas source arrangement can comprise a reservoir which contains inhalatory respiratory gas and can be emptied selectively through the flow-modification device. Likewise additionally or alternatively, the respiratory gas source arrangement can comprise or be a coupling formation, such as for instance as part of a quick coupling which is known per se or of a coupling for gas-carrying lines, with which the ventilation device can be connected to a respiratory gas reservoir installed in a building, as is the case in numerous hospitals.

Fundamentally preferably, the same minute volume is used in the challenge ventilation operation and in the normal ventilation operation as a target variable of the ventilation. It should, however, not be ruled out that in the challenge ventilation operation, due to the challenge ventilation frequency being decreased compared with the normal ventilation frequency, a challenge minute volume is used which is reduced compared with the normal minute volume by 20%, preferably by 15%, more preferably by 10%, in order to prevent administration of excessively large tidal volumes due to the lengthened challenge gap interval.

In the present application the terms ‘frequency’ and ‘rate’ are used synonymously in the sense of occurrence per unit of time.

When the challenge ventilation operation starts, the frequency of the mechanical ventilation as challenge ventilation frequency is preferably reduced to a value from 40% to 60% of the value of the normal ventilation frequency set during the normal ventilation operation. In absolute numbers, the reduced challenge ventilation frequency can by way of example be reduced to a value of about 6 to 8 breaths per minute, where the value of the frequency of the mechanical ventilation depends on the physical and clinical condition of the patient.

Frequency and gap interval are reciprocally inverses to one another.

The starting condition for the beginning of a challenge ventilation operation, namely the recognition of a plurality of spontaneous respiratory efforts occurring during the predetermined monitoring time period, can be realized in different ways. According to a first possible embodiment, the control device can during the predetermined monitoring time period capture breaths actually triggered by the patient. The number of actual patient-triggered breaths recognized in the predetermined monitoring time period can then be used as the number of spontaneous respiratory efforts occurring during the predetermined monitoring time period. To the number of actual patient-triggered breaths recognized in the predetermined monitoring time period there is equivalent a recognized spontaneous respiration frequency or spontaneous respiration rate, as the case may be, as patient-triggered breaths per minute or generally per unit of time.

A breath actually triggered by the patient can for example be recognized by the temporal course of the respiratory gas pressure. If the patient triggers a breath, then through the spontaneous respiratory effort of the patient which occurs before the moment of the next planned machine trigger, the respiratory gas pressure drops briefly in the temporal end region of the exhalation phase. This pressure drop normally does not happen with machine-triggered breaths.

Additionally or alternatively, a breath triggered by the patient triggered breath can be recognized by the temporal course of the respiratory gas flow, for instance if the magnitude of the inhalatory respiratory gas flow before the moment of the next planned machine trigger exceeds a predetermined threshold value. The threshold value can be set quantitatively low. It needs merely be so chosen that it makes possible, with sufficient reliability, differentiating a spontaneous respiratory effort from another short-term cause, for instance swallowing or a position change of the patient.

A triggering value of actual patient-triggered breaths recognized during the monitoring time period, on reaching which the challenge ventilation operation begins, can for example equal 4 to 6 patient-triggered breaths per minute. In trials, a value of 5 patient-triggered breaths per minute as a triggering value has proved to be especially advantageous.

The number of actual patient-triggered breaths per monitoring time period is normally smaller, even significantly smaller, than the spontaneous respiratory efforts actually occurring during the monitoring time period, since, as was described above, a plurality of spontaneous respiratory efforts of the patient can fall within the inhalation phase of an already machine-triggered breath and there simply be overridden by the control device. The recognized number of patient-triggered breaths per unit of time is indeed smaller than the number of actually occurring spontaneous respiratory efforts of the patient. However, patient-triggered breaths can be recognized with very high reliability and with very low errors. The overall lower number of capture events can be compensated for through a quantitatively appropriately low chosen triggering value.

Additionally or alternatively, for recognizing spontaneous respiratory efforts occurring during the predetermined monitoring time period, the control device can capture and analyze courses of the pressure of the inhalatory respiratory gas during the predetermined monitoring time period and/or courses of the flow of the inhalatory respiratory gas during the predetermined monitoring time period. An additional or alternative analysis of the exhalatory respiratory gas pressure and/or respiratory gas flow for recognizing spontaneous respiratory efforts is also conceivable in principle. Thus, only the inhalation phase or a part of same or only the exhalation phase or a part of same or the entire breath with inhalation and exhalation phase or a part of the entire breath with segments both of the inhalation phase and of the exhalation phase with the pressure course occurring therein and/or the flow course occurring therein can be used for recognizing spontaneous respiratory efforts occurring during the predetermined monitoring time period.

In the event of an attempted recognition of spontaneous respiratory efforts instead of actually patient-triggered breaths, the value recognized by the control device lies nearer to the real value of the actually occurring spontaneous respiratory efforts. However, their correct capturing is more complex than the capturing only of the patient-triggered breaths or their rate. Usually, a spontaneous respiratory effort during an inhalation phase of an already machine-triggered breath leads to characteristic changes in the course of the inhalatory respiratory gas pressure as a function of time and/or in the course of the inhalatory respiratory gas flow as a function of time, compared with an inhalation process undisturbed by a spontaneous respiratory effort. Through analysis of the temporal courses of the pressure of the inhalatory respiratory gas and/or of the flow of the inhalatory respiratory gas, spontaneous respiratory efforts can also be recognized during an already proceeding breath.

The spontaneous respiratory efforts thus recognized can also, when during the monitoring time period they quantitatively reach a predetermined triggering value, lead to the start of the challenge ventilation operation. The predetermined triggering value for the breaths recognized on the basis of the mentioned temporal value courses of the inhalatory respiratory gas is preferably quantitatively a different triggering value from that for the recognized actually patient-triggered breaths.

Additionally or alternatively, the sensor arrangement can comprise a sensor which captures an activity of the respiratory musculature of the patient, thus making possible the recognition of spontaneous respiratory efforts through capture of the activity of the respiratory musculature needed for this. Conceivable here is the capture of the esophageal pressure or the performing of electromyography on a muscle playing a part in the respiratory system of the patient. The number of the respiratory musculature activities captured during the monitoring time period can also be used by the control device as the number of recognized spontaneous respiratory efforts during the monitoring time period, in order to decide on their basis on the beginning of the challenge ventilation operation. A triggering value based on an activity of the respiratory musculature, upon reaching which the challenge ventilation operation is initiated, can exhibit a quantitatively different value from the aforementioned triggering values.

Especially the options for the capturing of spontaneous respiratory efforts of the patient which are based on the temporal value courses can be used at the ventilation device without additional sensor costs. To this end, it can for example be provided that the control device infers a spontaneous respiratory effort of the patient if the pressure of the inhalatory respiratory gas drops during an inhalation phase and rises again and/or if the flow of the inhalatory respiratory gas drops during an inhalation phase and rises again.

The temporal course of the flow of inhalatory respiratory gas when spontaneous respiratory effort of the ventilated patient occurs meanwhile, usually exhibits a bimodal course, i.e. a course with two local extreme values. The temporal course of the pressure of inhalatory respiratory gas exhibits, when a spontaneous respiratory effort occurs during the inhalation phase, a characteristic short-term pressure drop. When recognizing spontaneous respiration activity, the control device can search for such characteristic course sections.

By the term ‘flow’ the present application designates both a respiratory gas volume moved per unit of time and accordingly a respiratory gas volume flow and a respiratory gas mass moved per unit of time and accordingly a respiratory gas mass flow.

It is not the objective of the present invention to induce the spontaneous respiration of the patient, but rather to impede as little as possible any spontaneous respiration of the artificially ventilated patient. Through the still as before utilized gap interval, after whose expiration a breath is triggered by the control device, a sufficient supply of the patient with respiratory gas is also ensured during the challenge ventilation operation. However, it can be the case that during the challenge ventilation operation the supply of the patient with respiratory gas is quantitatively lower than in the normal ventilation operation. This can, as a positive side-effect, encourage the patient to maintain the spontaneous respiration.

In order to avoid an undersupply of the patient with respiratory gas through the challenge ventilation operation, the control device is configured to end again the challenge ventilation operation under defined conditions and to return to the normal ventilation operation.

The control device can, in order to avoid an undersupply of the patient, to be configured to end the challenge ventilation operation and to return to the normal ventilation operation, using the normal gap interval instead of the challenge gap interval, if at least one of the following conditions is met:
a) A number of breaths triggered spontaneously by the patient in a trigger observation period is smaller than a predetermined triggering threshold value,
b) A quantity of inhalatory respiratory gas administered to the patient in a quantity observation period is smaller than a predetermined quantity threshold value.

Condition a) indicates that the patient, although he initially displayed sufficient spontaneous respiration activity in order to start the challenge ventilation operation, does not display durably sufficient spontaneous respiratory efforts in order to take over the triggering of breaths on the basis of his natural and accordingly neural respiratory activity at a specified minimum rate. For example, the patient can temporarily display sufficient spontaneous respiration activity, however the latter is not stable.

To a number of breaths triggered spontaneously by the patient in a trigger observation period there is equivalent a corresponding rate and accordingly frequency of patient-triggered breaths. The latter too, has the unit breaths per unit of time.

Condition b) indicates simply that the patient has received, during the challenge ventilation operation, less respiratory gas administered than the predetermined quantity threshold value would allocate to him. This can, for example, be because the patient does indeed trigger breaths spontaneously, but his natural and accordingly neural gap interval is only slightly shorter or has become shorter, as the case may be, than the challenge gap interval. The patient does indeed, with every spontaneously triggered breath, supported by the ventilation device, receive a tidal volume set for him, but the administered tidal volumes can, with an excessively large gap interval, lead to smaller minute volumes than are envisaged for the patient. An excessively long neural gap interval or the longer challenge gap interval cannot be compensated for or only to a limited degree through higher tidal volumes, since only a certain quantity of respiratory gas can be administered to the patient per breath without harming him in the medium or long term.

Often when condition a) is met, sooner or later, depending on the choice of triggering threshold value on the one hand and of quantity threshold value on the other, condition b) will also be met and vice versa.

In further specification of the termination conditions, according to an advantageous development of the present invention condition a) can be met if at least one of the following sub-conditions is met:
a-i) After the beginning of the challenge ventilation operation the patient does not trigger a breath within the challenge gap interval as a first trigger observation period,
a-ii) At least one predetermined number of breaths during a second trigger observation period or at least one predetermined quota of breaths during a third trigger observation period is machine-triggered,
a-iii) A difference between a total breath rate captured during a fourth trigger observation period as the number of breaths captured overall in the fourth trigger observation period, divided by the duration of the fourth trigger observation period, and a target breath rate set in the preceding normal ventilation operation, is greater than a predetermined boundary breath rate difference.

Sub-condition a-i) concerns the case that the patient does indeed effect, on the basis of spontaneous respiratory efforts recognized by the control device, a start of the challenge ventilation operation, but the fulfilment of the conditions needed to this end goes back either to faulty capturing of spontaneous respiratory efforts or to a singular phase of spontaneous respiratory efforts. If the patient, after the start of the challenge ventilation operation, does not even trigger the first breath himself, the challenge ventilation operation is ended again. The challenge gap interval is then the first trigger observation period, since after its expiration the control device triggers the first breath after the beginning of the challenge ventilation operation and as from this moment the patient no longer has a possibility of triggering the first breath himself through spontaneous respiration activity.

Sub-condition a-ii) is a weaker version of sub-condition a-i) for the case that the patient does indeed trigger the first breath himself after the beginning of the challenge ventilation operation, but overall displays too low a spontaneous respiration activity to take over meaningfully the control of his ventilation on the basis of his neural ventilation frequency. Both the second and the third trigger observation period are each longer than the first trigger observation period. For example, the second and/or the third trigger observation period can comprise the duration of a predetermined number of breaths, for example five breaths. The second and the third trigger observation periods can exhibit equal or different durations. Thus the control device can, for example, return from the challenge ventilation operation into the normal ventilation operation if two or more out of five breaths during the challenge ventilation operation are machine-triggered or if 20% or more of the breaths during the challenge ventilation operation are machine-triggered, as the case may be.

Sub-condition a-iii) aims at an assessment of the natural and accordingly neural breathing frequency, also referred to as ‘breath rate’. If the latter is too low, the challenge ventilation operation is likewise ended. A total breath rate or total breathing frequency respectively is formed by adding the breaths triggered overall during the fourth trigger observation period, regardless of whether they are machine-triggered or patient-triggered, to a total number of breaths. This total number can be divided by the duration of the fourth trigger observation period to form a total breath rate.

The target breath rate or target breathing frequency respectively of the preceding normal ventilation operation is normally the normal breath rate or normal breathing frequency respectively, as the inverse of the normal gap interval. It is therefore a measure for a quantitatively correct ventilation of the relevant patient.

The difference between the total breath rate or total breathing frequency respectively and the target breath rate or target breathing frequency respectively of the preceding normal ventilation operation is thus a measure of whether the patient is sufficiently ventilated. Since in the challenge ventilation operation the ventilation of the patient should predominantly be patient-triggered, this difference is also a measure for assessing whether the spontaneous respiration activity of the patient is sufficient. If the total breath rate lies quantitatively further away from the target breath rate or target breathing frequency respectively of the preceding normal ventilation operation than by the predetermined boundary breath rate difference or boundary frequency difference respectively, then consequently absent sufficient spontaneous respiration activity the challenge ventilation operation is ended again. The fourth trigger observation period can exhibit a duration in the single-digit minute range, for instance 3 minutes, 2 minutes, or 1 minute. The boundary breath rate difference can for example be 2 to 4, preferably 3, breaths per minute.

In contrast to condition a) which is aimed at the spontaneous respiration activity, condition b) is aimed at the quantity of administered inhalatory respiratory gas. According to an advantageous development of the present invention, condition b) can be met if at least one of the following sub-conditions is met:
b-i) The minute volume administered to the patient is, for a first quantity observation period, smaller than a first boundary fraction of the minute volume set,
b-ii) The minute volume administered to the patient is, for a second quantity observation period, smaller than a second boundary fraction of the minute volume set, where the second quantity observation period is greater than the first quantity observation period and where the second boundary fraction value is greater than the first boundary fraction value.

Sub-conditions b-i) and b-ii) focus on different durations of quantity observation periods. Sub-condition b-i) allows for a shorter duration a stronger falling below the set minute volume actually to be administered. Sub-condition b-ii), which is designed rather for a longer quantity observation period, makes sure that over a longer time period there is only slight falling below the minute volume to be administered.

The first boundary fraction can for example be 70% to 80%, preferably 75%. The first quantity observation period can preferably lie in the range from 25 seconds to 1 minute, especially preferably from 30 seconds.

The second boundary fraction can be 85% to 95%, preferably 90%. The second quantity observation period can for example be 3 to 6 minutes, preferably 5 minutes.

Finally, the control device can be configured to end the challenge ventilation operation and to return to the normal ventilation operation, using the normal gap interval instead of the challenge gap interval, if for a predetermined challenge duration the rate or frequency respectively of breaths triggered spontaneously by the patient is greater than the target breath rate or target breathing frequency respectively of the preceding normal ventilation operation. This target breath rate is normally the normal breath rate, i.e. the inverse of the normal gap interval. Alternatively expressed, the control device can be configured to end the challenge ventilation operation and to return to the normal ventilation operation, using the normal gap interval instead of the challenge gap interval, if for a predetermined challenge duration the neural gap interval of the patient is shorter than the normal gap interval of the preceding normal ventilation operation. In this case the challenge ventilation operation is ended successfully. This is because then the problematic initial situation is no longer present, whereupon the neural gap interval as the inverse of the rate or frequency respectively of breaths triggered spontaneously by the patient is longer than the normal gap interval of the normal ventilation operation. The predetermined challenge duration can for example be 20 to 45 minutes, preferably 30 minutes.

In order to prevent a challenge ventilation operation being started again too soon after an ended challenge ventilation operation, the control device can be configured to begin, after the end of a preceding challenge ventilation operation, a following further challenge ventilation operation at the earliest after expiration of a predetermined waiting time. The predetermined waiting time can be 5 to 20 minutes, preferably 10 minutes. In this way it can be ensured that the patient does not have to take over again the control of his artificial ventilation through his neural breathing frequency if he has proved only a short while ago to be incapable of same.

It can further be conceived that the predetermined waiting time increases with the number of ended challenge ventilation operation phases, in order to allow the patient enough time to stabilize his spontaneous respiration activity, in order eventually to be able to trigger breaths at his neural breathing frequency in a further challenge ventilation operation phase.

The concept of the gap interval, as used in the present application preferably as the inverse of a breathing frequency, extends from the triggering moment of a preceding breath up to the triggering moment of the immediately subsequent following breath. It should, however, not be ruled out that gap intervals are used which are not an inverse of a breathing frequency but rather which extend for example from the end moment of an inhalation phase of the preceding breath up to the start moment of the inhalation phase of the following breath.

The ventilation device can exhibit a CO₂ sensor for capturing CO₂ in the respiratory gas, in particular in the exhalatory respiratory gas, which is connected in a signal-transmitting manner with the control device. The control device is preferably configured to ascertain from the data of the CO₂ sensor a value for CO₂ content, in particular the end tidal CO₂ content, in the exhalatory respiratory gas of the patient. Such a value, measured for instance in the last 20%, preferably in the last 10%, especially preferably in the last 5%, of the duration of the exhalation phase between cycling and triggering, is especially meaningful since the then sensorially captured respiratory gas is no longer or only still to a negligible degree mixed with CO₂-free respiratory gas from a dead space volume of the respiratory gas line arrangement. If the CO₂ content ascertained in the respiratory gas, in particular in the exhalatory respiratory gas, exceeds a predetermined threshold value, this can indicate that the ventilation of the patient depends to an excessive degree on the ventilation device and the patient is not capable of breathing himself. The control device can therefore be configured not to change over into the challenge ventilation operation when the ascertained CO₂ content does not exceed the predetermined threshold value, despite conditions otherwise being met for a changeover into the challenge ventilation operation.

These and other objects, aspects, features and advantages of the invention will become apparent to those skilled in the art upon a reading of the Detailed Description of the invention set forth below taken together with the drawings which will be described in the next section.

### BRIEF DESCRIPTION OF THE DRAWINGS

The invention may take physical form in certain parts and arrangement of parts, a preferred embodiment of which will be described in detail and illustrated in the accompanying drawings which forms a part hereof and wherein:

FIG. 1A schematic depiction of a ventilation device according to the invention prepared for artificial ventilation of a patient,

FIG. 2A rough schematic depiction of the problem underlying the present invention,

FIG. 3A depiction of ventilation parameter courses of a successful changeover from a normal ventilation operation into a challenge ventilation operation, and

FIG. 4A depiction of ventilation parameter courses of a changeover from a normal ventilation operation into a challenge ventilation operation which shortly thereafter is discontinued again.

### DESCRIPTION OF PREFERRED EMBODIMENTS

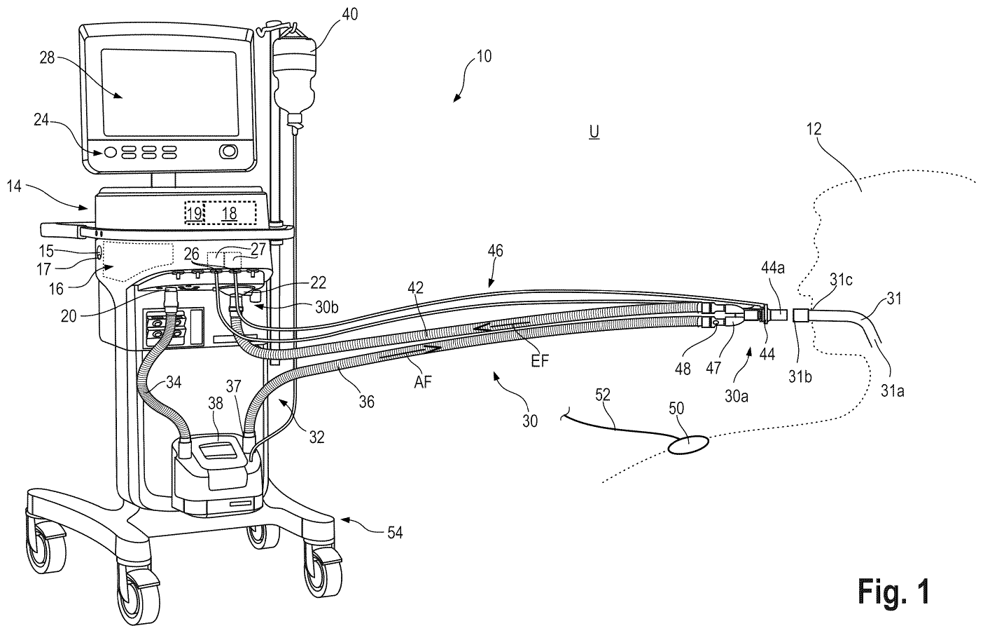

Referring now to the drawings wherein the showings are for the purpose of illustrating preferred and alternative embodiments of the invention only and not for the purpose of limiting the same, in FIG. 1, an embodiment of a ventilation device according to the invention is labelled generally by 10. The ventilation device 10 serves in the depicted example for the artificial ventilation of a human patient 12.

The ventilation device 10 exhibits a housing 14 in which an intake port 15 is configured and—not recognizable from outside because of the opaque housing material—a flow-modification device 16 and a control device 18 are accommodated. The intake port 15 allows the flow-modification device 16 to aspirate ambient air from the outside environment U of the ventilation device and after purification which is known per se to supply it through at least one filter as respiratory gas to the patient 12. The intake port 15 is therefore a respiratory gas source arrangement within the meaning of the present application.

In the intake port 15 there can be situated an ambient temperature sensor 17 which measures the temperature of the air of the environment U and transmits it to the control device 18.

The flow-modification device 16 is constructed in a manner which is known per se and can exhibit a pump, a compressor, a fan, a pressure tank, a reducing valve and the like. The ventilation device 10 further exhibits in a manner which is known per se an inhalation valve 20 and an exhalation valve 22.

The control device 18 is usually realized as a computer or microprocessor. It comprises a data store labelled 19 in FIG. 1, in order to be able to store and if required to retrieve data needed for the operation of the ventilation device 10. The data store 19 can also, in the case of network operation, be situated outside the housing 14 and be connected with the control device 18 through a data-transmission link. The data-transmission link can be formed by a cable or radio link. In order, however, to prevent data-transmission link interference being able to impact the operation of the ventilation device 10, the data store 19 is preferably integrated into the control device 18 or at least accommodated in the same housing 14 as the latter.

For the input of data into the ventilation device 10 or more precisely into the control device 18, the ventilation device 10 can exhibit an input device 24 which in the example depicted in FIG. 1 is represented by a keyboard. As will still be elucidated further below, the keyboard is not necessarily the only data input of the control device 18. In fact, additionally or alternatively to the keyboard the control device 18 can receive data over various data inputs, for instance over a network cable, a radio link, or over sensor connectors 26.

For the output of data to the treating therapists, the ventilation device 10 can exhibit an output device 28, in the depicted example a screen.

For artificial ventilation, the patient 12 is connected with the ventilation device 10, more precisely with the flow-modification device 16 in the housing 14, via a respiratory gas line arrangement 30. To this end, the patient 12 is intubated by means of an endotracheal tube as a patient interface 31. Unlike the depicted example, the patient interface can be formed by a mask. A proximal longitudinal end 31a of the patient interface 31 delivers the inhalatory respiratory gas flow AF into the lung of the patient 12. Through the proximal longitudinal end 31a there also flows the exhalatory respiratory gas flow EF into the respiratory gas line arrangement 30.

A distal longitudinal end 31b of the patient interface 31 is configured for connecting with the respiratory gas line arrangement 30. From the point 31c downstream in the inhalation direction up to the proximal longitudinal end 31a, the patient interface is surrounded by the body of the patient 12. This means conversely that the patient interface 31 from its distal longitudinal end 31b up to the point 31c is exposed to the outside environment U and is in predominantly convective heat-exchange contact with it.

The respiratory gas line arrangement 30 exhibits an inhalation hose 32 via which fresh respiratory gas can be conducted from the flow-modification device 16 into the lung of the patient 12. The inhalation hose 32 can be interrupted and exhibit a first inhalation hose 34 and a second inhalation hose 36, between which a humidification device 38 for targeted humidification and where applicable also temperature control of the inhalatory respiratory gas supplied to the patient 12 can be provided. The humidification device 38 can be connected with an external fluid reservoir 40, via which water for humidification or also a medication, for instance for inhibiting inflammation or for dilating the respiratory tract, can be added to the humidification device 38. When using the present ventilation device 10 as an anesthesia ventilation device, volatile anesthetics can in this manner be administered to the patient 12 in a controlled way via the ventilation device 10. The humidification device 38 makes sure that the fresh respiratory gas is supplied to the patient 12 at a predetermined humidity, where applicable with the addition of a medication aerosol, and at a predetermined temperature.

The second inhalation hose 36 is heatable electrically in the present example through a line heating device 37. The line heating device 37 can be actuated for operation through the control device 18. The above notwithstanding, the first inhalation hose 34 can also be heatable and/or the at least one hose 34 and/or 36 be heatable through a line heating device 37 which is other than electric, for instance through flushing with a heat-exchange medium.

The respiratory gas line arrangement 30 further exhibits besides the already mentioned inhalation valve 20 and exhalation valve 22 an exhalation hose 42, via which metabolized respiratory gas is discharged from the lung of the patient 12 into the outside environment U as exhalatory respiratory gas flow EF.

At the distal longitudinal end 30b of the respiratory gas line arrangement 30, the inhalation hose 32 is coupled with the inhalation valve 20 and the exhalation hose 42 with the exhalation valve 22. Out of the two valves, preferably only one is always opened at the same time for letting through a gas flow. The actuation control of the valves 20 and 22 takes place likewise through the control device 18.

During a ventilation cycle, initially for the duration of the inhalation phase the exhalation valve 22 is closed and the inhalation valve 20 opened, such that fresh inhalatory respiratory gas can be conducted from the housing 14 to the patient 12. A flow of the fresh respiratory gas is effected through a targeted pressure increase of the respiratory gas through the flow-modification device 16. Due to the pressure increase, the fresh respiratory gas flows into the lung of the patient 12 and expands there the lung-adjacent body region, i.e. in particular the chest, against the individual elasticity of the lung-adjacent organs. Hereby the gas pressure inside the lung of the patient 12 also rises.

At the end of the inhalation phase, the inhalation valve 20 is closed and the exhalation valve 22 opened. The exhalation phase begins. Due to the elevated gas pressure, until the end of the inhalation phase, of the respiratory gas located in the lung of the patient 12, the latter flows after the opening of the exhalation valve 22 into the outside environment U, whereby the gas pressure in the lung of the patient 12 decreases with progressing flow duration. Once the gas pressure in lung 12 reaches a positive end-exhalatory pressure (PEEP) set at the ventilation device 10, i.e. a slightly higher pressure than the atmospheric pressure, the exhalation phase is ended through closing of the exhalation valve 22 and there follows a further ventilation cycle.

During the inhalation phase, the patient 12 is supplied with what is referred to as a ventilation tidal volume, i.e. the respiratory gas volume per breath. The ventilation tidal volume multiplied by the number of ventilation cycles per minute, i.e. multiplied by the ventilation frequency, yields the minute volume of the present performed artificial ventilation.

The ventilation device 10, in particular the control device 18, is preferably configured to update and/or ascertain, as the case may be, repeatedly during the ventilation operation, ventilation operating parameters which distinguish the ventilation operation of the ventilation device 10, in order to make sure that at every moment the ventilation operation is matched as optimally as possible to the respective patient 12 to be ventilated. Especially advantageously, the determination of one or several ventilation operating parameters takes place at the ventilation frequency, such that for each ventilation cycle up-to-date and thus optimally to the patient 12 matched ventilation operating parameters can be provided.

To this end, the ventilation device 10 can be connected for data transmission with one or several sensors which monitor the state of the patient and/or the operation of the ventilation device 10. Merely by way of example for a series of possible sensors, let a proximal flow sensor 44 be mentioned in FIG. 1 which captures quantitatively the respiratory gas flow prevailing in the respiratory gas line arrangement 30, namely both the inhalatory respiratory gas flow AF and the exhalatory respiratory gas flow EF. The proximal flow sensor 44, preferably configured as a differential pressure sensor, can by means of a sensor line arrangement 46 be coupled with the data inputs 26 of the control device 18. The sensor line arrangement 46 can, but does not have to, comprise electric signal transmission lines. It can likewise exhibit hose lines which transmit the gas pressure prevailing in the flow direction on both sides of the flow sensor 44 to the data inputs 26, where these are quantified by pressure sensors 27.

More precisely, in the preferred embodiment example the respiratory gas line arrangement 30 exhibits at its proximal longitudinal end region 30a a separately configured Y-line section 47 which at its distal end region is connected with the second inhalation hose 36 and with the exhalation hose 42 and which at its proximal end region is connected with the proximal flow sensor 44.

The proximal flow sensor 44 exhibits at its proximal end region a coupling formation 44a, with which the patient interface 31, which instead of a tube could also be a mask, can be coupled with the proximal flow sensor 44 and consequently with the respiratory gas line arrangement 30.

The second inhalation hose 36 can exhibit at its proximal longitudinal end region a proximal temperature sensor 48 which measures the temperature of the respiratory gas flow AF in the second inhalation hose 36 as close as possible to the patient 12 and transmits it to the control device 18.

The sensor arrangement working together with the control device 18 can furthermore exhibit a diaphragm sensor 50 which is connected with the control device 18 in a signal-transmitting manner via a data line 52. The diaphragm sensor 50 can capture activity of the respiratory musculature of the patient 12 and transmit appropriate information to the control device 18. From the signals of the diaphragm sensor 50 and/or from the signals of the pressure sensors 27, the control device 18 can ascertain spontaneous respiratory efforts of the patient. From the signals of the pressure sensors 27, there are ascertained in the case of the differential pressure flow sensor 44 being used here flow signals which quantify the inhalatory respiratory gas flow AF and the exhalatory respiratory gas flow EF respectively.

Solely for the sake of completeness let it be noted that the ventilation device 10 according to the invention can be accommodated as a mobile ventilation device 10 on a rolling rack 54.

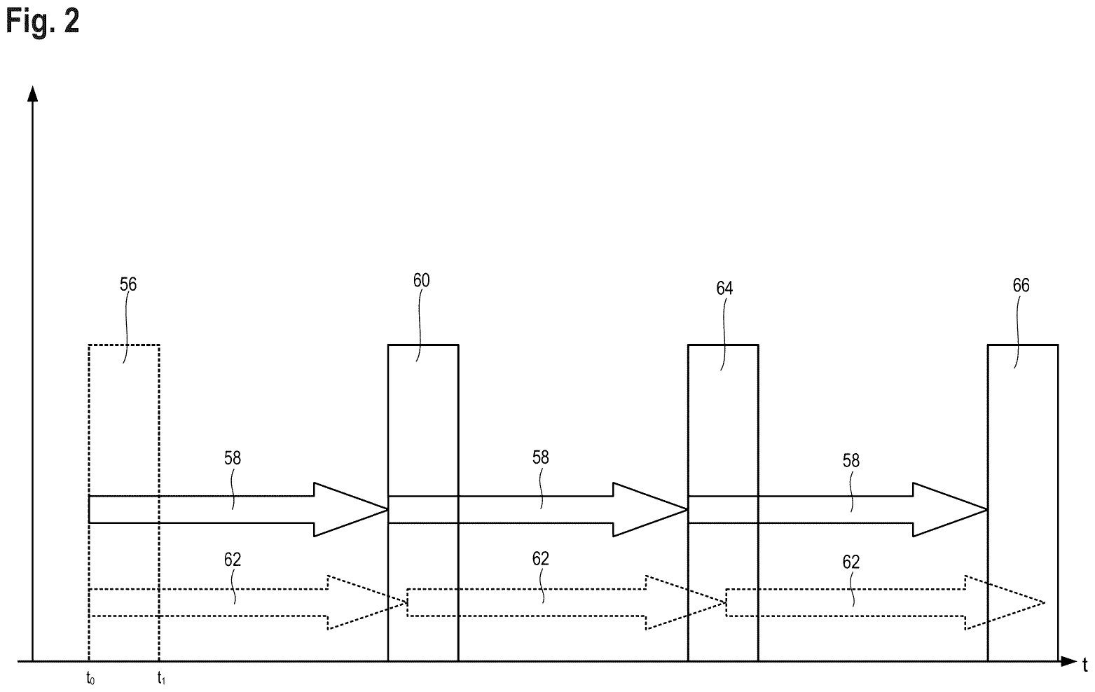

In FIG. 2 there is depicted in rough schematic form the problem underlying the present invention. The abscissa of FIG. 2 is a time axis. An inhalation process triggered spontaneously by the patient is depicted in rough schematic form by a dotted line 56. The spontaneously- or rather patient-triggered inhalation process 56 begins at the moment to and ends at the moment t₁.

An arrow 58 is depicted in FIG. 2 by a solid line, representing the applicable normal gap interval. After expiration of the normal gap interval 58, the control device 18 triggers a following breath with a following inhalation process 60. As a machine-triggered inhalation process 60, it is drawn with a solid line like the normal gap interval 58 as part of the control of the ventilation device 10.

In contrast, the natural or neural gap interval 62 is depicted in FIG. 2 by a dotted line, after the expiration of which the patient 12, due to his physical and health-related constitution, would trigger a following inhalation process. Since the neural gap interval 62 is slightly longer than the normal gap interval 58 of the control device 18, the patient 12 has in the first instance no opportunity of triggering breaths himself, since the end of his relevant neural gap interval 62, at least for the following three depicted following inhalation processes 60, 64, and 66 always lies in an already running machine-triggered inhalation process, thus any spontaneous respiratory effort of the patient 12 is simply overridden by the control device 18.

In order to alleviate or prevent the situation in which a patient actually capable of triggering is overridden, through a disadvantageous constellation of mechanical and personal circumstances, by the control device 18 or by the ventilation device 10 as the case may be, the control device 18 starts, under predetermined conditions such as a captured predetermined plurality and/or rate respectively of spontaneous respiration activities of the patient 12, a challenge ventilation operation. In this challenge ventilation operation, the challenge gap interval is decreased by about 40% to 60% compared with the normal gap interval in the normal ventilation operation.

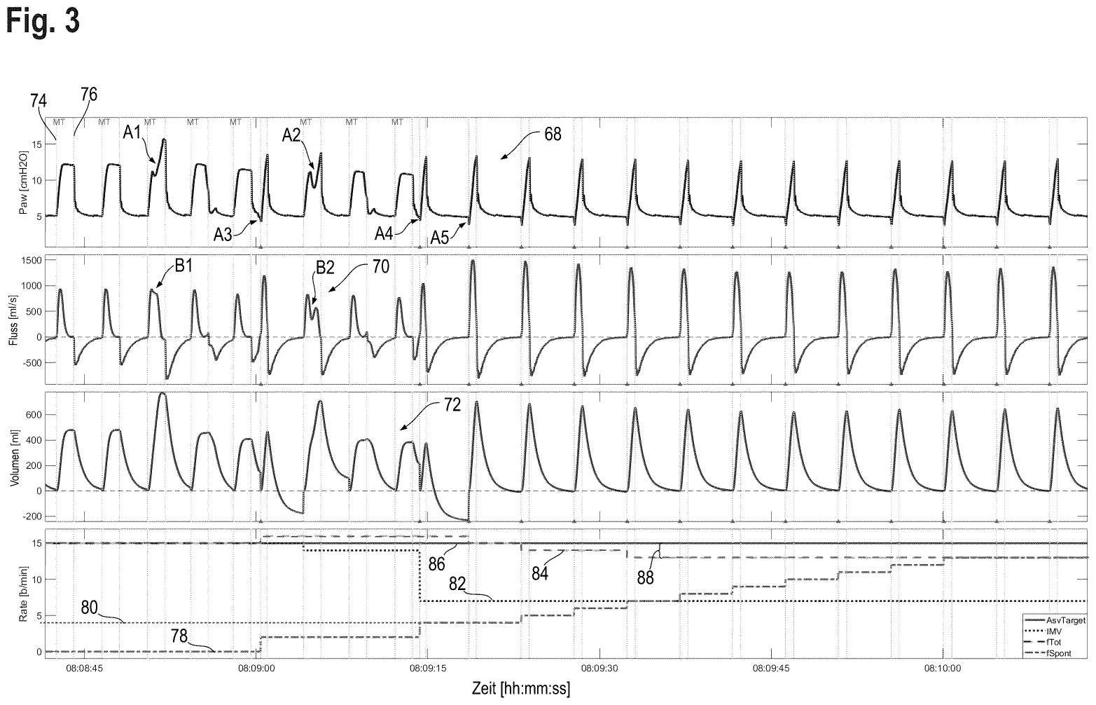

FIG. 3 shows by way of example parametrically such a case of a challenge ventilation operation following a normal ventilation operation. The abscissa of the graph in FIG. 3 is the time axis. Graph 68 in the top row of FIG. 3 shows the course of the pressure of the respiratory gas in cm-water column. The unit of pressure is not significant here, more important is the qualitative course of the pressure curve 68.

In the row below, graph 70 shows the flow of respiratory gas in milliliters per second, i.e. as volume flow. Positive flow values indicate an inhalatory respiratory gas flow AF, negative flow values an exhalatory respiratory gas flow EF.

In the third row, graph 72 shows the respiratory gas volume supplied to the patient 12. The respiratory gas volume is the breath-wise integral of the respiratory gas flow over time.

The pressure curve 68 shows at various places spontaneous respiratory efforts of the patient 12, for instance at A1 during the third depicted inhalation phase, or at A2 during the seventh depicted inhalation phase. In both cases, the pressure value first drops through the spontaneous respiratory effort of the patient 12 and then rises again.

Since the pressure of the respiratory gas is the driving cause establishing the respiratory gas flow, the spontaneous pressure drop based on spontaneous respiratory activity is also reflected in the flow curve 70, for instance at B1 in the third depicted inhalation phase, or at B2 during the seventh depicted inhalation phase. At B2 the flow curve 70 shows a pronounced bimodal course, which is typical for spontaneous respiration activities of an artificially ventilated patient during an inhalation phase.

In the present example, however, the control device 18 performs no analysis of the pressure curve 68 or of the flow curve 70 in order to ascertain spontaneous respiratory efforts of the patient 12. Instead, the control device 18 ascertains spontaneous breaths triggered by the patient 12. These are recognizable by a characteristic pressure drop at the end of the exhalation phase and/or respectively at the beginning of the following inhalation phase triggered through this pressure drop, and thereby before a following breath would be machine-triggered. Such a characteristic pressure drop can be recognized in FIG. 3 at the pressure curve 68, for example at A3, A4, and every breath following A4, see for example at A5.

In FIG. 3, the first vertical line 74 shows across all horizontal rows a triggering moment and the second vertical line 76 shows across all horizontal rows a cycling moment. The vertical lines following one another from left to right are always alternately trigger and cycling moments. Machine-triggered breaths exhibit in FIG. 3 at the top edge of the vertical triggering moment lines an “MT” label. In contrast, the triangles at the abscissa of curves 68, 70, and 72 each denote a patient-triggered breath.

At the spontaneous triggering event A4, let there be reached a predetermined plurality of spontaneously triggered breaths for a predetermined monitoring time period, for instance 1 minute or 3 minutes. The predetermined plurality of spontaneously triggered breaths reached in the predetermined monitoring time period corresponds in the depicted example to a spontaneous breathing frequency of four breaths per minute, which in the depicted example is the triggering threshold value for changing over from the normal ventilation operation into a challenge ventilation operation. Graph 78 in the bottom row of FIG. 3 displays the spontaneous breathing frequency of the patient 12 ascertained by the control device 18 at each current moment. Line 80 indicates the boundary value or more specifically triggering threshold value for the initiation of the challenge ventilation operation. The spontaneous breathing frequency or respiration rate is ascertained in the present example over a predetermined number of contiguous breaths, for example over 8 breaths. The spontaneous breathing frequency is calculated from the number of ascertained spontaneously triggered breaths, multiplied by the quotient of 60 seconds divided by the duration of eight breaths in seconds.

Graph 82 in FIG. 3, bottom row, indicates the normal ventilation frequency at which the patient 12 will continue to be ventilated mechanically in a machine-triggered manner during the challenge ventilation operation if he himself would not display any spontaneous respiration activity whatsoever. When the spontaneous breathing frequency of the patient 12 in accordance with Graph 78 reaches the triggering threshold value, the control device 18 decreases the normal ventilation frequency in the depicted example by 50%, from 14 breaths per minute to a challenge ventilation frequency of 7 breaths per minute. Other patients can, due to their physical condition and state of health, receive other values.

The normal ventilation frequency is the inverse of the previously discussed normal gap interval. Through the lowering of the normal ventilation frequency, which corresponds to increasing the gap interval by the inverse of the factor applied to the normal ventilation frequency, the patient 12 now has the possibility of triggering breaths based on his neural breathing frequency and thus taking over the control of the ventilation device 10 as regards the triggering of breaths. In the case of a successful challenge ventilation operation, the artificial ventilation of the patient 12 then takes place in better accord with his natural respiratory behavior than when continuing the normal ventilation operation.

Graph 84 shows the total ventilation frequency ascertained by the control device 18, i.e. the sum of mechanically- and patient-triggered breaths per unit of time. The control device 18 determines Graph 84 as well as the spontaneous breathing frequency of Graph 78 as a sliding frequency or rate value, where the relative temporal position of the captured breaths to the predetermined monitoring time period sliding along with it can effect a slight modification of the calculated total ventilation frequency, without the respiratory behavior of the patient 12 changing significantly.

Graph 86 displays the normal ventilation frequency set in the normal ventilation operation as the target ventilation frequency for an adaptively supporting ventilation operation. In this adaptively supporting ventilation operation as the normal ventilation operation, the patient is ventilated at the normal ventilation frequency as a target ventilation frequency. He can, however, also trigger himself through spontaneous respiration activities.

The control device 18 regulates the ventilation operation in the depicted example on the basis of periods 5 breaths each and thereby on the basis of s different periodicity than the periodicity for ascertaining the spontaneous breathing frequency.

The challenge ventilation operation remains a supporting ventilation operation, however the number of machine-triggered breaths is reduced through quantitatively decreasing the normal ventilation frequency to the challenge ventilation frequency.

After the start of the challenge ventilation operation in FIG. 3, the patient 12 triggers each individual breath himself, namely at a sufficient spontaneous breathing frequency for a predetermined trigger observation period, such that the difference between the total ventilation frequency according to Graph 84 and the normal or target ventilation frequency as the case may be set in the preceding normal ventilation operation according to Graph 86 is quantitatively smaller than a predetermined boundary frequency difference 88. The boundary frequency difference only just tolerated can for example be 3 breaths per minute.

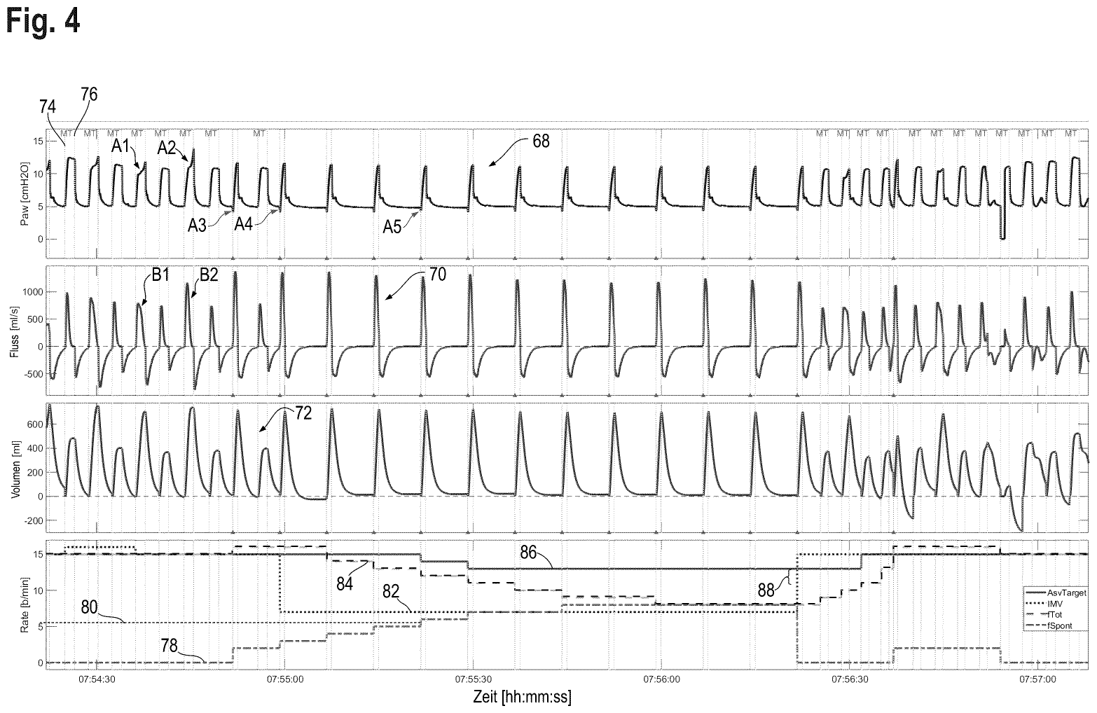

In FIG. 4 there are depicted the same Graphs 68 to 86 and the same boundary frequency difference 88 in a different ventilation process.

The predetermined triggering threshold value according to line 80 in FIG. 4 is reached shortly before 7:55:00, whereupon the challenge ventilation operation is started by the normal ventilation frequency according to Graph 82 is decreased in the depicted example by slightly more than 50% from 15 breaths per minute to a challenge ventilation frequency of 7 breaths per minute.

Although from the beginning of the challenge ventilation operation of FIG. 4 the patient 12 initially triggers each following breath himself, the challenge ventilation operation is ended again since the neural breathing frequency of the patient is too low and consequently according to Graph 84 the total ventilation frequency ascertained by the control device 18 differs for the duration of a predetermined trigger observation period by more than the maximum permissible boundary frequency difference 88.

At the end of the challenge ventilation operation in FIG. 4, the challenge ventilation frequency is reset according to Graph 82 again to its old value as normal ventilation frequency.

A renewed start of a challenge ventilation operation can, after its end, only take place after the elapsing of a predetermined waiting time, which preferably lasts at least 10 minutes.

While considerable emphasis has been placed on the preferred embodiments of the invention illustrated and described herein, it will be appreciated that other embodiments, and equivalences thereof, can be made and that many changes can be made in the preferred embodiments without departing from the principles of the invention. Furthermore, the embodiments described above can be combined to form yet other embodiments of the invention of this application. Accordingly, it is to be distinctly understood that the foregoing descriptive matter is to be interpreted merely as illustrative of the invention and not as a limitation.

1-13. (canceled)

14. A ventilation device for a ventilation operation for mechanical administration of respiratory gas to a patient, where the ventilation device comprises:
a respiratory gas source arrangement which provides an inhalatory respiratory gas for administration to the patient,
a flow-modification device which is configured to generate and/or quantitatively modify an inhalatory respiratory gas flow,
a respiratory gas line arrangement in order to convey the inhalatory respiratory gas flow from the respiratory gas source arrangement towards the patient,
a sensor arrangement which is configured to capture at least one state variable of the inhalatory respiratory gas flow and/or a mass flow of the inhalatory respiratory gas flow and/or a respiratory musculature activity of the patient,
a control device with a data store, where the control device is connected with the data store and with the sensor arrangement for signal transmission, and where the control device is configured for controlling the flow-modification device,
where the control device is configured to actuate, in a normal ventilation operation, the flow-modification device for initiating a machine-triggered breath starting from a preceding breath not before expiration of a predetermined temporal normal gap interval for generating an inhalatory respiratory gas flow, in order to effect a machine-triggered following breath which immediately follows the preceding breath,
where the control device is further configured to recognize, on the basis of data ascertained by the sensor arrangement, a spontaneous respiratory effort of the patient during the ventilation operation of the ventilation device,
where the control device is further configured, when in a predetermined monitoring time period it has recognized at least one predetermined plurality of spontaneous respiratory efforts of the patient, to change over into a challenge ventilation operation in which instead of the normal gap interval a challenge gap interval is used which is temporally longer compared with the normal gap interval, where in the challenge ventilation operation the control device not before expiration of the challenge gap interval effects a machine-triggered following breath which immediately follows a preceding breath.

15. The ventilation device according to claim 14, wherein the control device is configured to supply to the patient at least in the challenge ventilation operation, upon a spontaneous trigger of the patient, under predetermined ventilation conditions, inhalatory respiratory gas.

16. The ventilation device according to claim 15, wherein in the challenge ventilation operation and in the normal ventilation operation the same minute volume is used as a target variable of the ventilation.

17. The ventilation device according to claim 14, wherein in the challenge ventilation operation and in the normal ventilation operation the same minute volume is used as a target variable of the ventilation.

18. The ventilation device according to claim 14, wherein the control device for recognizing spontaneous respiratory efforts occurring during the predetermined monitoring time period captures breaths actually triggered by the patient during the predetermined monitoring time period.

19. The ventilation device according to claim 14, wherein the control device for recognizing spontaneous respiratory efforts occurring during the predetermined monitoring time period captures courses of the pressure of the inhalatory and/or of the exhalatory respiratory gas during the predetermined monitoring time period and/or courses of the flow of the inhalatory respiratory gas during the predetermined monitoring time period.

20. The ventilation device according to claim 19, wherein the control device infers a spontaneous respiratory effort of the patient if the pressure of the inhalatory respiratory gas drops during an inhalation phase and rises again and/or if the flow of the inhalatory respiratory gas drops during an inhalation phase and rises again.

21. The ventilation device according to claim 14, wherein the control device ends the challenge ventilation operation and returns to the normal ventilation operation, using the normal gap interval instead of the challenge gap interval, if at least one of the following conditions is met:
condition a) a number of breaths triggered spontaneously by the patient in a trigger observation period is smaller than a predetermined triggering threshold value,
condition b) a quantity of inhalatory respiratory gas administered to the patient in a quantity observation period is smaller than a predetermined quantity threshold value.

22. The ventilation device according to claim 21, wherein condition a) is met if at least one of the following sub-conditions is met:
sub-condition a-i) after the beginning of the challenge ventilation operation the patient does not trigger a breath within the challenge gap interval as a first trigger observation period,
sub-condition a-ii) at least one predetermined number of breaths during a second trigger observation period or at least one predetermined quota of breaths during a third trigger observation period is machine-triggered,
sub-condition a-iii) a difference between a total breathing frequency captured during a fourth trigger observation period as the number of breaths captured overall in the fourth trigger observation period overall, divided by the duration of the fourth trigger observation period, and a target breathing frequency set in the preceding normal ventilation operation, is greater than a predetermined boundary frequency difference.

23. The ventilation device according to claim 22, wherein condition b) is met if at least one of the following sub-conditions is met:
sub-condition b-i) the minute volume administered to the patient is, for a first quantity observation period, smaller than a first boundary fraction of the minute volume set,
sub-condition b-ii) the minute volume administered to the patient is, for a second quantity observation period, smaller than a second boundary fraction of the minute volume set, where the second quantity observation period is greater than the first quantity observation period and where the second boundary fraction value is greater than the first boundary fraction value.

24. The ventilation device according to claim 21, wherein condition b) is met if at least one of the following sub-conditions is met:
sub-condition b-i) the minute volume administered to the patient is, for a first quantity observation period, smaller than a first boundary fraction of the minute volume set,
sub-condition b-ii) the minute volume administered to the patient is, for a second quantity observation period, smaller than a second boundary fraction of the minute volume set, where the second quantity observation period is greater than the first quantity observation period and where the second boundary fraction value is greater than the first boundary fraction value.

25. The ventilation device according to claim 14, wherein the control device ends the challenge ventilation operation and returns to the normal ventilation operation, using the normal gap interval instead of the challenge gap interval, if for a predetermined challenge duration the frequency of breaths triggered spontaneously by the patient is greater than the target breathing frequency set in the preceding normal ventilation operation.

26. The ventilation device according to claim 14, wherein the control device is configured to begin, after the end of a preceding challenge ventilation operation, a following further challenge ventilation operation at the earliest after expiration of a predetermined waiting time.

27. The ventilation device according to claim 26, wherein the predetermined waiting time increases with the number of ended challenge ventilation operation phases.

28. A ventilation method for mechanical administration of respiratory gas to a patient, where the ventilation method comprises the following steps:
effecting a machine-triggered following breath which immediately follows a preceding breath by generating an inhalatory respiratory gas flow by means of a flow-modification device starting from the preceding breath after expiration of a predetermined temporal normal gap interval,
capturing at least one state variable of the inhalatory respiratory gas flow and/or of a mass flow of the inhalatory respiratory gas flow and/or of a respiratory musculature activity of the patient through a sensor arrangement,
recognizing a spontaneous respiratory effort of the patient during the ventilation operation of the ventilation device on the basis of data ascertained by the sensor arrangement,
where the ventilation method comprises the following further steps:
when in a predetermined monitoring time period at least one predetermined plurality of spontaneous respiratory efforts of the patient has been recognized, changing over into a challenge ventilation operation in which instead of the normal gap interval a challenge gap interval is used which is temporally longer compared with the normal gap interval temporal,
effecting a machine-triggered following breath which immediately follows a preceding breath after expiration of the challenge gap interval.
